(19) United States
(12) Patent Application Publication
(10) Pub. No.: US 2017/0303774 A1
(43) Pub. Date: Oct. 26, 2017

(54) ENDOSCOPE MAGNIFICATION OPTICAL SYSTEM AND ENDOSCOPE

(71) Applicant: HOYA CORPORATION, Tokyo (JP)

(72) Inventor: Sachiko NASU, Tokyo (JP)

(73) Assignee: HOYA CORPORATION, Tokyo (JP)

(21) Appl. No.: 15/513,399

(22) PCT Filed: Aug. 29, 2016
(86) PCT No.: PCT/JP2016/075157

§ 371 Date: Mar. 22, 2017

(30) Foreign Application Priority Data
Sep. 7, 2015 (JP) 2015-176173

(51) Int. Cl. A61B 1/00; G02B 15/15

(52) U.S. Cl. CPC A61B 1/00188; A61B 1/00096; G02B 15/15

(57) ABSTRACT

An endoscope magnification optical system including: a first lens group that has positive power and includes at least a negative lens with a concave surface facing an object side, a positive lens with a convex surface facing an image side, and a doublet obtained by bonding a negative lens and a positive lens; a second lens group that has negative power and includes at least a doublet obtained by bonding a negative lens and a positive lens; and a third lens group that has positive power and includes at least a positive lens and a doublet obtained by bonding a negative lens and a positive lens, is configured such that predetermined conditions are satisfied.

### TECHNICAL FIELD

The present invention relates to an endoscope magnification optical system and an endoscope in which an endoscope magnification optical system is incorporated.

### BACKGROUND ART

In the field of medicine, endoscopes (fiberscopes or electronic scopes) are commonly known as devices for observing the interior of a body cavity of a patient, and are provided for practical use. In order to observe abnormalities in detail, some of these types of endoscopes are equipped with a magnification optical system having a magnification function.

With an endoscope magnification optical system according to Japanese Patent 5580956 (hereinafter written as “Patent Document 1”), a first lens group having positive power, a second lens group having negative power, and a third lens group having positive power are arranged in the stated order starting from the object side, and by correcting aberrations in the lens groups having the positive power, a change in the aberrations caused by magnification is suppressed.

### SUMMARY OF INVENTION

However, with the lens configuration described as an example in Patent Document 1, if an attempt is made to ensure a magnification that is sufficient for observation of the interior of a body cavity using an endoscope while giving consideration to control during magnification, a large movement amount of a movable lens group (second lenses) during magnification needs to be ensured, and therefore a problem is indicated in that it is difficult to reduce the size of the endoscope.

The present invention has been made in light of the foregoing circumstances, and it is an object, thereof to provide an endoscope magnification optical system and an endoscope that are suitable for a smaller design, while ensuring a movement amount that is needed for control during magnification for a movable lens group and ensuring a magnification that is sufficient for observation of the interior of a body cavity using an endoscope.

An endoscope magnification optical system according to an embodiment of the present invention includes, in order starting from an object side, a first lens group having positive power, a second lens group having negative power, and a third lens group having positive power, and is configured to magnify an optical image by moving at least the second lens group in an optical axis direction with respect to the first lens group, which is a fixed lens group. The first lens group includes at least, in order starting from the object side, a negative lens with a concave surface facing an image side, a positive lens with a convex surface facing the image side, and a doublet obtained by bonding a negative lens and a positive lens. The second lens group includes a doublet obtained by bonding a negative lens and a positive lens, and includes at least a negative lens and a positive lens in alignment in the stated order starting from the object side, or a positive lens and a negative lens in alignment in the stated order starting from the object side. The third lens group includes at least, in order starting from the object side, a positive lens and a doublet obtained by bonding a negative lens and a positive lens.

In the case where an interval between the first lens group and the second lens group at the telephoto end is defined as D₁ₜ (unit: mm), an interval between the first lens group and the second lens group at the wide angle end is defined as D_{1w} (unit: mm), a maximum image height is defined as y (unit: mm), a composite focal length from the first lens group to the third lens group at the telephoto end is defined as fₜ (unit: mm) and a composite focal length from the first lens group to the third lens group at the wide angle end is defined as f_{w} (unit: mm), the endoscope magnification optical system according to an embodiment of the present invention satisfies the following two conditional expressions:

0.43<(D₁ₜ−D_{1w})/y<0.70

120<fₜ/f_{w}<1.45

In the case where a focal length of the negative lens located the closest to the object of the first lens group is defined as f₀₁ (unit: mm) and a focal length of the second lens group is defined as f₂ (unit: mm), the endoscope magnification optical system according to an embodiment of the present invention may have a configuration in which the following conditional expression:

0.6<f₀₁/f₂<1.0

is satisfied.

Also, the endoscope magnification optical system according to an embodiment of the present invention may be configured such that the following conditional expression:

0.45<(D₁ₜ−D_{1w})/f_{w}<0.75

is satisfied.

Also, the endoscope magnification optical system according to an embodiment of the present invention may be configured such that the following conditional expression:

0.3<SF₂<4.0

where SF₂:(rₛ₁+rₛ₂)/(rₛ₁−rₛ₂)
rₛ₁: object-side curvature radius of the positive lens in the first lens group (unit: mm)
rₛ₂: image-plane-side curvature radius of the positive lens in the first lens group (unit: mm)
   is satisfied.

Also, in the case where the incidence angle at the maximum image height y at the wide angle end on the object-side surface of the negative lens located the closest to the object of the first lens group is defined as θ (unit: degree), the endoscope magnification optical system according to an embodiment of the present invention may be configured such that the following conditional expression:

θ≦75°

is satisfied.

Also, the endoscope magnification optical system according to an embodiment of the present invention may have a configuration in which an aperture configured to move integrally with the second lens group on the optical axis is included between the first and the second lens groups.

Also, an endoscope according to an embodiment of the present invention is a device in which the above-described endoscope magnification optical system is mounted on a leading end thereof.

According to an embodiment of the present invention, an endoscope magnification optical system and an endoscope that are suitable for a smaller design are provided while a movement amount that is needed for control during magnification is ensured for a movable lens group and a magnification that is sufficient for observing the interior of the body cavity using the endoscope is ensured.

### BRIEF DESCRIPTION OF DRAWINGS

FIG. 1 is an external view showing an exterior of an electronic scope according to an embodiment of the present invention.

FIG. 2 is a lens arrangement diagram showing a configuration of an endoscope magnification optical system according to Working Example 1 of the present invention.

FIG. 3 is a diagram showing various aberrations in an endoscope magnification optical system according to Working Example 1 of the present invention.

FIG. 4 is a lens arrangement diagram showing a configuration of an endoscope magnification optical system according to Working Example 2 of the present invention.

FIG. 5 is a diagram showing various aberrations in an endoscope magnification optical system according to Working Example 2 of the present invention.

FIG. 6 is a lens arrangement diagram showing a configuration of an endoscope magnification optical system according to Working Example 3 of the present invention.

FIG. 7 is a diagram showing various aberrations in an endoscope magnification optical system according to Working Example 3 of the present invention.

FIG. 8 is a lens arrangement diagram showing a configuration of an endoscope magnification optical system according to Working Example 4 of the present invention.

FIG. 9 is a diagram showing various aberrations in an endoscope magnification optical system according to Working Example 4 of the present invention.

FIG. 10 is a lens arrangement diagram showing a configuration of an endoscope magnification optical system according to Working Example 5 of the present invention.

FIG. 11 is a diagram showing various aberrations of an endoscope magnification optical system according to Working Example 5 of the present invention.

### DESCRIPTION OF EMBODIMENTS

Hereinafter, an endoscope magnification optical system according to an embodiment of the present invention and an electronic scope including an endoscope magnification optical system will be described with reference to the drawings.

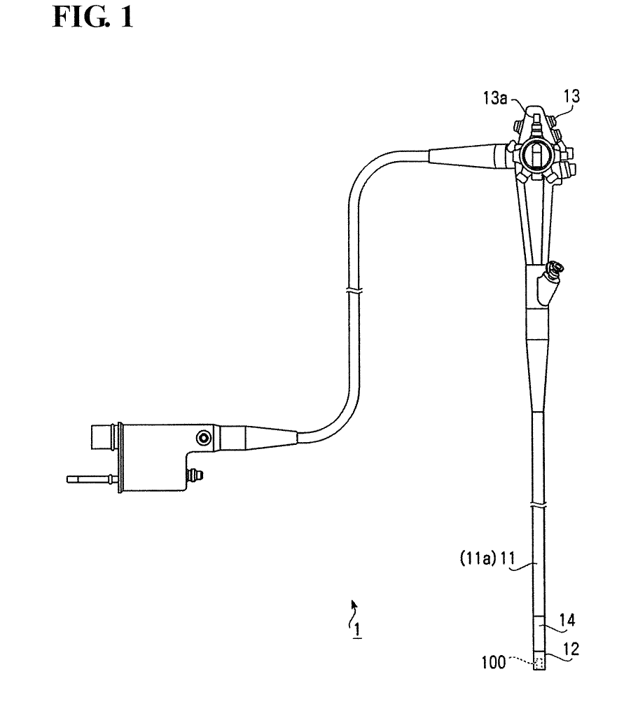

FIG. 1 is an external view showing an exterior of an electronic scope 1 according to an embodiment of the present invention. As shown in FIG. 1, the electronic scope 1 includes an insertion portion flexible tube 11 that is covered by a flexible sheath 11a. A leading end portion (bending portion 14) of the insertion portion flexible tube 11 bends in response to a remote operation (specifically, an operation of rotating a bending operation knob 13a) from a hand operation portion 13 coupled to a base end of the insertion portion flexible tube 11. The bending mechanism is a known mechanism incorporated in a common endoscope, and the bending mechanism causes the bending portion 14 to bend by pulling an operation wire linked to the rotation operation of the bending operation knob 13a. A base end of a leading end portion 12 covered by a housing made of hard resin is coupled to the leading end of the bending portion 14. The direction of the leading end portion 12 changes according to the bending operation performed through the rotation operation of the curving operation knob 13a, and thus a region imaged by the electronic scope 1 moves.

An endoscope magnification optical system 100 (the block indicated by diagonal lines in FIG. 1) is incorporated in the interior of the housing made of resin of the leading end portion 12. The endoscope magnification optical system 100 allows light from an object in the imaging region to form an image on a light receiving plane of a solid image sensor (not shown) in order to obtain image data of the object.. Examples of the solid image sensor include a CCD (Charge Coupled Device) image sensor or a CMOS (Complementary Metal Oxide Semiconductor) image sensor.

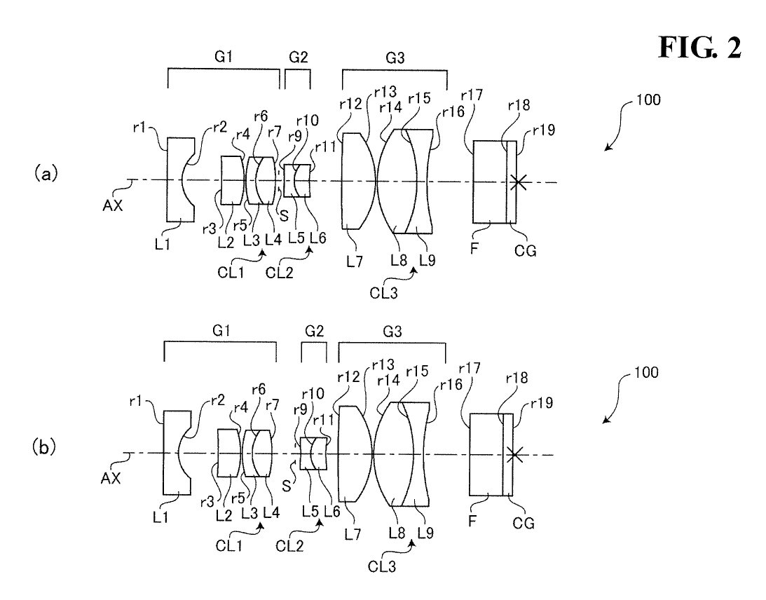

FIG. 2(a) is a cross-sectional view showing the endoscope magnification optical system 100 according to Working Example 1 (to be described in detail later) of the present invention and an arrangement of optical components arranged downstream thereof. Next, FIG. 2(a) will be used as a reference to give a detailed description of the endoscope magnification optical system 100 according to an embodiment of the present invention.

As shown in FIG. 2(a), the endoscope magnification optical system 100 includes, in order starting from the object side, a first lens group G1 having positive power, an aperture S, a second lens group G2 having negative power, and a third lens group G3 having positive power. The optical lenses constituting the lens groups G1 to G3 have shapes with rotational symmetry centered on an optical axis AX of the endoscope magnification optical system 100. A color correction filter F for a solid image sensor is arranged downstream of the third lens group G3. The color correction filter F is adhered to a cover glass CG that protects a solid image sensor.

The first lens group G1 is a lens group that has positive power and is arranged on the object side relative to the aperture S. In order starting from the object side, the first lens group G1 includes at least a negative lens L1 with a concave surface facing the image side, a positive lens L2 with a convex surface facing the image side, and a doublet CL1 obtained by bonding a negative lens L3 and a positive lens L4. The expression “includes at least” is used because a configuration example in which another optical element such as a parallel plate is additionally arranged is also possible in the scope of the technical idea of the present invention. The expression “includes at least” is used for similar reasons in the description of the second lens group G2 and the third lens group G3 as well.

The second lens group G2 is a lens group having negative power and in order to suppress a change in a chromatic aberration, includes at least a doublet CL2 obtained by bonding a negative lens L5 and a positive lens LB. In FIG. 2, in the doublet CL2, the negative lens L5 is arranged on the object side and the positive lens L6 is arranged on the image side, but in another embodiment, the positive lens may be arranged on the object side and the negative lens may be arranged on the image side (for example, see later-described Working Example 2 of the present invention). The second lens group G2 moves integrally with the aperture S in the optical axis AX direction in order to magnify the optical image formed on the image receiving plane of the solid image sensor. By integrally moving the second lens group G2 and the aperture S, the occurrence of astigmatism when at the telephoto end is effectively suppressed.

The third lens group G3 is a lens group having positive power, and includes at least, in order starting from the object side, a positive lens L7, and a doublet CL3 obtained by bonding a negative lens L8 and a positive lens L9. The positive lens L7 is arranged mainly for the purpose of correcting a spherical aberration, and the doublet CL3 is arranged mainly for the purpose of correcting a chromatic aberration of magnification.

The aperture S is a plate-shaped member having a predetermined circular opening centered on the optical axis AX, or is a light-blocking film that coats the lens surface located the closest to the aperture S of the second lens group G2 (in the configuration example shown in FIG. 2(a), surface r9 on the object side of the negative lens L5), excluding a predetermined circular region centered on the optical axis AX. The thickness of the aperture S is very thin compared to the thicknesses of the optical lenses constituting the endoscope magnification optical system 100 and may be ignored when calculating the optical performance of the endoscope magnification optical system 100. For this reason, in the present specification, the thickness of the aperture S is considered to be zero in the following description.

In the case where the interval between the first lens group G1 and the second lens group G2 on the telephoto end is defined as D₁ₜ (unit: mm), the interval between the first lens group G1 and the second lens group G2 at the wide angle end is defined as D_{1w} (unit: mm), the maximum image height is defined as y (unit: mm), the focal length of the entire system at the telephoto end (composite focal length from the first lens group G1 to the third lens group) is defined as fₜ (unit: mm), and the focal length of the entire system at the wide angle end is defined as fₜ (unit: mm), the endoscope magnification optical system 100 has, a configuration in which the following conditional expressions (1) and (2):

0.43<(D₁ₜ−D_{1w})/y<0.70 (1)

1.20<fₜ/f_{w}<1.45 (2)

are satisfied.

Conditional expression (1) defines the ratio between the movement amount and the maximum image height y of the second lens group G2. Due to conditional expression (1) being satisfied, a sufficient movement amount of the second lens group G2 during magnification is ensured and thus control during magnification is simplified, and it is possible to design a smaller endoscope magnification optical system 100 while ensuring a magnification that is sufficient for observation of the interior of a body cavity using the electronic scope 1.

If the value in the middle of conditional expression (1) is greater than or equal to the value on the right side, the movement amount of the second lens group G2 will be too large, and therefore it will be difficult to keep the overall length of the endoscope magnification optical system 100 short and it will be difficult to design a smaller endoscope magnification optical system 100.

If the value in the middle of conditional expression (1) is less than or equal to the value on the left side, it will be possible to keep the entire length of the endoscope magnification optical system 100 short, but control during magnification will be difficult clue to the fact that the movement amount of the second lens group G2 will be small, and the outer diameter will be larger due to the fact that the image height will be larger.

Conditional expression (2) defines the ratio between the focal length of the entire system at the wide angle end and the focal length of the entire system at the telephoto end. Due to conditional expression (2) being satisfied, the enlargement ratio of the object can be kept in a suitable range relative to the typical observation distance when performing enlarged observation inside of a body cavity using an electronic scope 1 (e.g., in the case of performing image capture at a position located slightly away from a pipe wall or the like in the body cavity).

If the value in the middle of conditional expression (2) is greater than or equal to the value on the right side, the resolution during enlarged observation (on the telephoto end) will decrease due to the change in the F number accompanying change in the magnification increasing.

If the value in the middle of conditional expression (2) is less than or equal to the value on the left side, the magnification during enlarged observation (magnification on the telephoto end) cannot be sufficiently ensured.

Also, in the case where the focal length of the negative lens located the closest to the object of the first lens group G1 (in the example shown in FIG. 2, the negative lens L1) is defined as f₀₁ (unit: mm) and the focal length of the second lens group G2 is defined as f₂ (unit: mm), the endoscope magnification optical system 100 has a configuration in which the following conditional expression (3):

0.6<f₀₁/f₂<1.0 (3)

is satisfied.

Conditional expression (3) defines the ratio between the focal length of the negative lens L1 and the focal length of the second lens group G2. Due to conditional expression (3) being satisfied, the effective flux radius of the first lens group G1 is suppressed, which is advantageous for reducing the size of the endoscope magnification optical system 100.

If the value in the middle of conditional expression (3) is greater than or equal to the value on the right side, the negative power of the second lens group G2, which is the movable lens group, will increase, and as compensation for the fact that the movement amount of the second lens group G2 can be kept small, the negative power of the negative lens L1 will become too weak, the effective F number during enlarged observation (on the telephoto end) will increase, and the resolution will decrease.

If the value in the middle of conditional expression (3) is less than or equal to the value on the left side, the negative power of the negative lens L1 will increase and the effective flux radius of the first lens group G1 will be suppressed, but a comatic aberration will increase in size, the negative power of the second lens group G2 will decrease, and the movement amount of the second lens group G2 will increase, which is not advantageous for reducing the size of the endoscope magnification optical system 100.

Also, the endoscope magnification optical system 100 has a configuration in which the following conditional expression (4):

0.45<(D₁ₜ−D_{1w})/f_{w}<0.75 (4)

is satisfied.

Conditional expression (4) defines the ratio between the movement amount of the second lens group G2 and the focal length of the entire system at the wide angle end. Due to conditional expression (4) being satisfied, the size of the endoscope magnification optical system 100 can be reduced, and a sufficient movement amount of the second lens group G2 during magnification can be ensured, which simplifies control during magnification.

If the value in the middle of conditional expression (4) is greater than or equal to the value on the right side, the movement amount of the second lens group G2 will increase, which is not advantageous for reducing the size of the endoscope magnification optical system 100.

If the value in the middle of conditional expression (4) is less than or equal to the value on the left side, it is possible to keep the entire length of the endoscope magnification optical system 100 short, but control during magnification will be difficult due to the fact that the movement amount of the second lens group G2 will be small.

Also, the endoscope magnification optical system 100 has a configuration in which the following conditional expression (5)

0.3<SF₂<4.0 (5)

where SF₂: (rₛ₁+rₛ₂)/(rₛ₁−rₛ₂)
rₛ₁: object-side curvature radius of positive lens in first lens group G1 (unit: mm)
rₛ₂: image-plane-side curvature radius of positive lens in first lens group G1 (unit: mm)
   is satisfied.

Conditional expression (5) defines the shape of the positive lens (in the example shown in FIG. 2, the positive lens L2) in the first lens group G1. Due to conditional expression (5) being satisfied, the eccentric sensitivity (amount of change in aberrations when eccentricity occurs in the arrangement plane or shape plane with respect to the optical axis AX, for example) in the first lens group G1 is reduced.

In the case where the value in the middle of conditional expression (5) is greater than or equal to the value on the right side and in the case where the value in the middle is less than or equal to the value on the left side, the emission angle of the light from the upstream lens (in the example shown in FIG. 2, the positive lens L2) to the doublet CL1 will increase, and the eccentric sensitivity in the first lens group G1 will increase.

Also, in the case where the incidence angle at the maximum image height y on the object-side surface (hereinafter referred to as “surface closest to the object” for convenience in the description) of the negative lens located the closest to the object (in the example shown in FIG. 2, the negative lens L1) of the first lens group G1 is defined as θ (unit: degree), the endoscope magnification optical system 100 has a configuration in which the following conditional expression (6):

θ≦75° (6)

is satisfied.

Conditional expression (6) defines the incidence angle at the maximum image height y at the wide angle end on the surface closest to the object in the endoscope magnification optical system 100. In general, no anti-reflection coating is applied to the surface closest to the object in the endoscope. In the endoscope magnification optical system 100 according to an embodiment of the present invention, clue to conditional expression (6) being satisfied, a decrease in the light amount caused by surface reflection is suppressed.

In the case where conditional expression (6) is not satisfied, if the negative lens located the closest to the object of the first lens group G1 is formed of a highly-refractive glass material with a refractive index exceeding 1.8, the surface reflectance will exceed around 30%, and therefore a prominent decrease in the light amount occurs.

Next, five specific numerical working examples of the above-described endoscope magnification optical system 100 will be described. The endoscope magnification optical system 100 according to the numerical working examples 1 to 5 is arranged in the leading end portion 12 of the electronic scope 1 shown in FIG. 1.

### WORKING EXAMPLE 1

As described above, the configuration of the endoscope magnification optical system 100 according to Working Example 1 of the present invention is as shown in FIG. 2(a). FIG. 2(a) is a cross-sectional view showing a lens arrangement when the magnification position is at the wide angle end. FIG. 2(b) shows a cross-sectional view showing the lens arrangement when the magnification position is at the telephoto end.

The specific numerical configuration (setting values) of the endoscope magnification optical system 100 (and the optical components arranged downstream thereof) according to the present Working Example 1 are shown in Table 1. The surface numbers NO shown in Table 1 each correspond to a surface reference number rn (n being a natural number) in FIG. 2, except for surface number 8, which corresponds to the aperture S. In Table 1. R (unit mm) indicates the curvature radii of the surfaces of the optical members. D (unit: mm) indicates the optical member thicknesses or the optical member intervals on the optical axis AX, N(d) indicates the refractive indexes at the d-line (wavelength 588 nm), and vd indicates the Abbe number at the d-line.

Table 2 shows the specifications (effective F number, entire system focal length (unit: mm), optical magnification, half field angle (unit: degree), image height (unit: mm), group interval D7 (unit.: mm), and group interval D11 (unit: mm)) of the endoscope magnification optical system 100 according to the present Working Example 1, for both the wide angle end and the telephoto end. The group interval D7 is the group interval between the first lens group G1 and the second lens group G2. The group interval D11 is the group interval between the second lens group G2 and the third lens group G3. The group interval D7 and the group interval D11 change according to the magnification position.

TABLE 1
Working Example 1
(surface data)

| NO | R | D | N(d) | νd |
|---|---|---|---|---|
| 1 | INFINITY | 0.371 | 1.88300 | 40.8 |
| 2 | 0.853 | 0.987 | | |
| 3 | −10.382 | 0.560 | 1.51633 | 64.1 |
| 4 | −2.012 | 0.041 | | |
| 5 | 2.090 | 0.248 | 1.84666 | 23.8 |
| 6 | 1.020 | 0.491 | 1.77250 | 49.6 |
| 7 | −2.480 | D 7 | | |
| 8 Aperture | INFINITY | 0.136 | | |
| 9 | −1.943 | 0.248 | 1.80400 | 46.6 |
| 10 | 0.660 | 0.379 | 1.69895 | 30.1 |
| 11 | 3.513 | D11 | | |
| 12 | 25.864 | 0.847 | 1.72916 | 54.7 |
| 13 | −2.010 | 0.041 | | |
| 14 | 2.309 | 0.990 | 1.77250 | 49.6 |
| 15 | −3.096 | 0.248 | 1.92286 | 18.9 |
| 16 | 4.761 | 1.153 | | |
| 17 | INFINITY | 0.825 | 1.51407 | 73.4 |
| 18 | INFINITY | 0.248 | 1.51000 | 64.1 |
| 19 | INFINITY | — | | |

TABLE 2
Working Example 1 (various types of data)

| | Wide angle | Telephoto |
|---|---|---|
| F number | 6.7 | 7.9 |
| Focal length | 1.00 | 1.30 |
| Magnification | −0.111 | −0.575 |
| Half field angle | 76.0 | 38.0 |
| Image height | 0.97 | 0.97 |
| D7 | 0.083 | 0.582 |
| D11 | 0.800 | 0.301 |

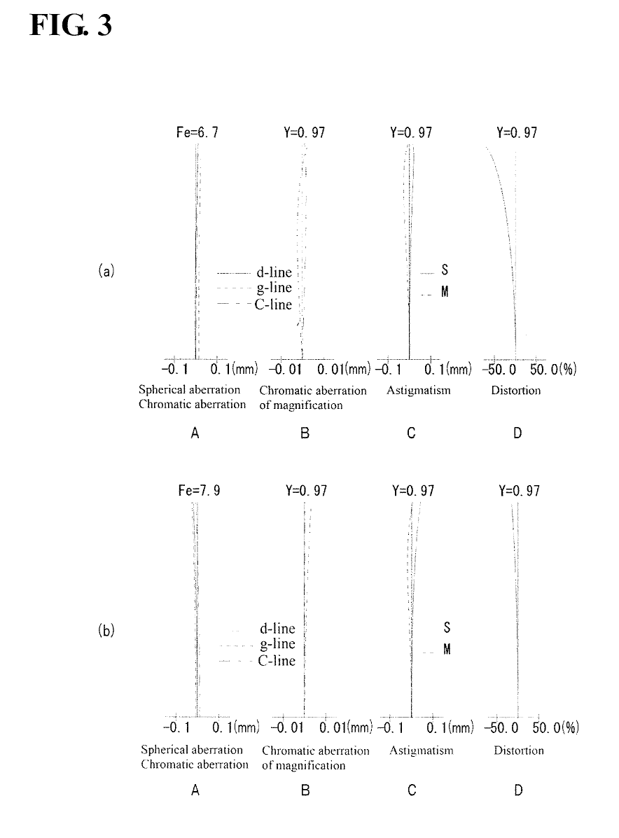

Graphs A to D in FIG. 3(a) are diagrams of various aberrations at the time when the magnification position is at the wide angle end in the endoscope magnification optical system 100 according to the present Working Example 1. Graphs A to D in FIG. 3(b) are diagrams of various aberrations at the time when the magnification position is at the telephoto end in the endoscope magnification optical system 100 according to the present Working Example 1. Graphs A in FIGS. 3(a) and 3(b) show spherical aberrations and axial chromatic aberrations at the d-line, g-line (wavelength: 436 nm), and C-line (wavelength: 656 nm). Graphs B in FIGS. 3(a) and 3(b) show chromatic aberrations of magnification at the d-line, g-line, and C-line. In graphs A and B, the solid lines indicate aberrations at the d-line, the dotted lines indicate aberrations at the g-line, and the one-dot chain lines indicate aberrations at the C-line. Graphs C in FIGS. 3(a) and 3(b) show astigmatisms. In graphs C, the solid lines indicate sagittal components, and the dotted lines indicate meridional components. Graphs D in FIGS. 3(a) and 3(b) show distortion. The vertical axes of graphs A to C indicate the image height, and the horizontal axes indicate the aberration amount. The vertical axes of graphs D indicate the image height, and the horizontal axes indicate the distortion rate. Note that the description of the tables and diagrams of Working Example 1 also applies to the tables and diagrams presented in the following numerical working examples.

As can be understood from FIG. 2 and Tables 1 and 2, the endoscope magnification optical system 100 according to the present embodiment 1 is made smaller, while ensuring a sufficient movement amount of the second lens group G2 during magnification, making it easier to perform magnification control, and ensuring a magnification that is sufficient for observation of the interior of a body cavity using the electronic scope 1. Also, as shown in FIGS. 3(a) and 3(b), the aberrations are corrected favorably at both the wide angle end and the telephoto end. Note that in the central region between the wide angle end and the telephoto end, the various aberrations change within the ranges indicated by FIGS. 3(a) and 3(b). In other words, although the endoscope magnification optical system 100 according to Working Example 1 is small, magnification control is easy to perform, a magnification that is sufficient for observing the interior of a body cavity using the electronic scope 1 is ensured, and the optical performance is favorable at every magnification position between the wide angle end and the telephoto end.

### WORKING EXAMPLE 2

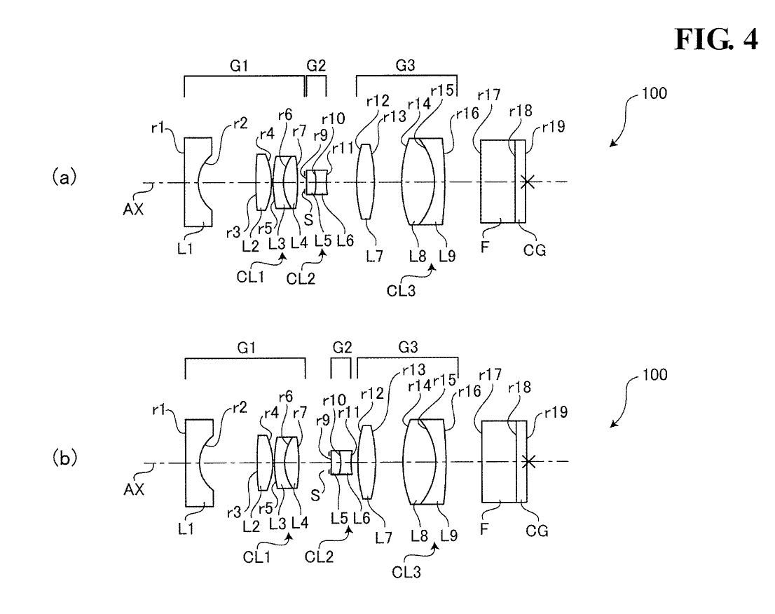

FIGS. 4(a) and 4(b) are cross-sectional views showing arrangements of optical components included in the endoscope magnification optical system 100 according to Working Example 2. FIG. 4(a) shows a lens arrangement for when the magnification position is at the wide angle end. FIG. 4(b) shows a lens arrangement for when the magnification position is at the telephoto end.

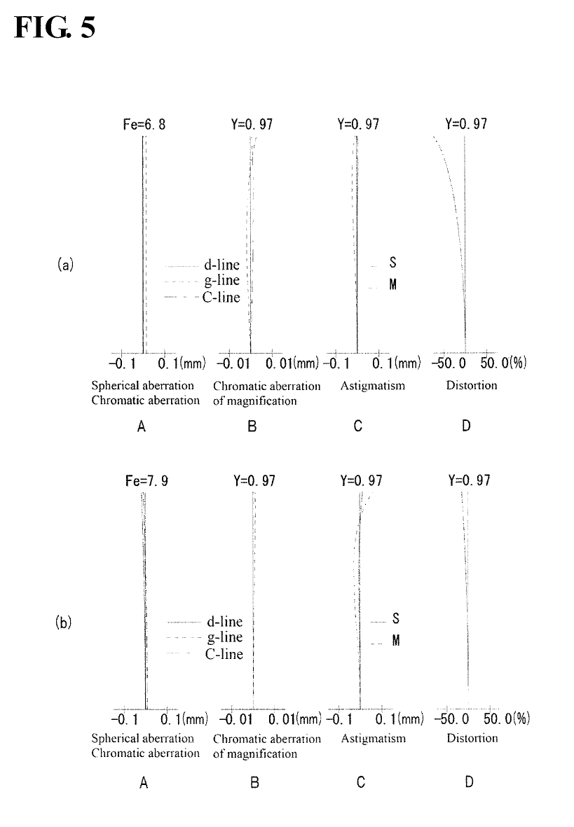

Graphs A to D in FIG. 5(a) are diagrams of various aberrations at the time when the magnification position is at the wide angle end in the endoscope magnification optical system 100 according to the present Working Example 2. Graphs A to D in FIG. 5(b) are diagrams of various aberrations at the time when the magnification position is at the telephoto end in the endoscope magnification optical system 100 according to the present Working Example 2.

Table 3 shows a specific numerical value configuration of the optical components included in the endoscope magnification optical system 100 according to the present Working Example 2, and Table 4 shows the specifications of the endoscope magnification optical system 100 according to the present Working Example 2. As can be understood from FIGS. 4 and 5 and Tables 3 and 4, although the endoscope magnification optical system 100 according to the present Working Example 2 is small, magnification control is easy to perform, a magnification that is sufficient for observing the interior of a body cavity using the electronic scope 1 is ensured, and the optical performance is favorable at every magnification position from the wide angle end to the telephoto end.

TABLE 3
Working Example 2
(surface data)

| NO | R | D | N(d) | νd |
|---|---|---|---|---|
| 1 | INFINITY | 0.331 | 1.88300 | 40.8 |
| 2 | 0.850 | 1.393 | | |
| 3 | 7.969 | 0.377 | 1.51633 | 64.1 |
| 4 | −1.662 | 0.041 | | |
| 5 | 3.009 | 0.248 | 1.84666 | 23.8 |
| 6 | 1.136 | 0.343 | 1.77250 | 49.6 |
| 7 | −4.550 | D 7 | | |
| 8 Aperture | INFINITY | 0.054 | | |
| 9 | −3.910 | 0.227 | 1.84666 | 23.8 |
| 10 | −1.098 | 0.248 | 1.80400 | 46.6 |
| 11 | 1.622 | D11 | | |
| 12 | 3.413 | 0.429 | 1.77250 | 49.6 |
| 13 | −3.756 | 0.661 | | |
| 14 | 3.078 | 0.791 | 1.72916 | 54.7 |
| 15 | −1.642 | 0.248 | 1.95906 | 17.5 |
| 16 | −9.171 | 0.867 | | |
| 17 | INFINITY | 0.828 | 1.51407 | 73.4 |
| 18 | INFINITY | 0.248 | 1.51000 | 64.1 |
| 19 | INFINITY | — | | |

TABLE 4
Working Example 2 (various types of data)

| | Wide angle | Telephoto |
|---|---|---|
| F number | 6.8 | 7.9 |
| Focal length | 1.00 | 1.28 |
| Magnification | −0.111 | −0.558 |
| Half field angle | 75.7 | 38.2 |
| Image height | 0.97 | 0.97 |
| D7 | 0.166 | 0.732 |
| D11 | 0.730 | 0.164 |

### WORKING EXAMPLE 3

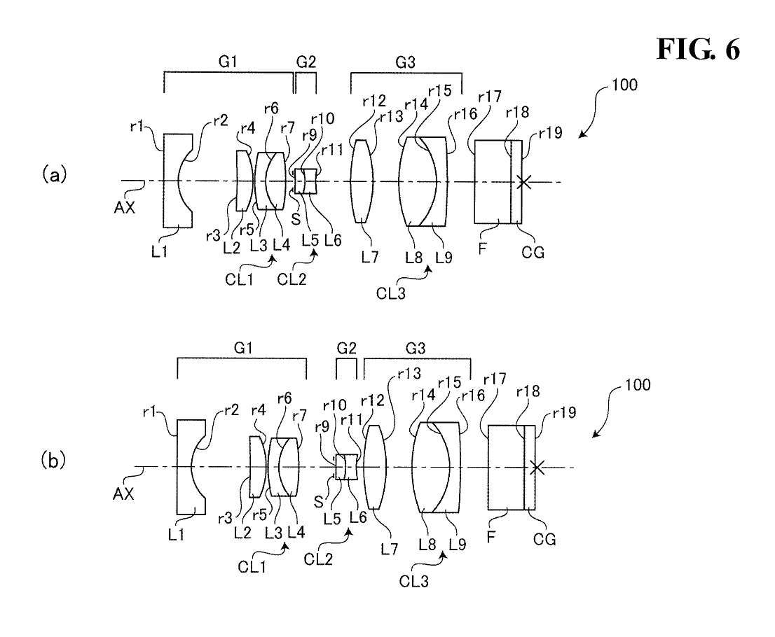

FIGS. 6(a) and 6(b) are cross-sectional views showing arrangements of optical components included in the endoscope magnification optical system 100 according to Working Example 3. FIG. 6(a) shows a lens arrangement for when the magnification position is at the wide angle end. FIG. 6(b) shows a lens arrangement for when the magnification position is at the telephoto end.

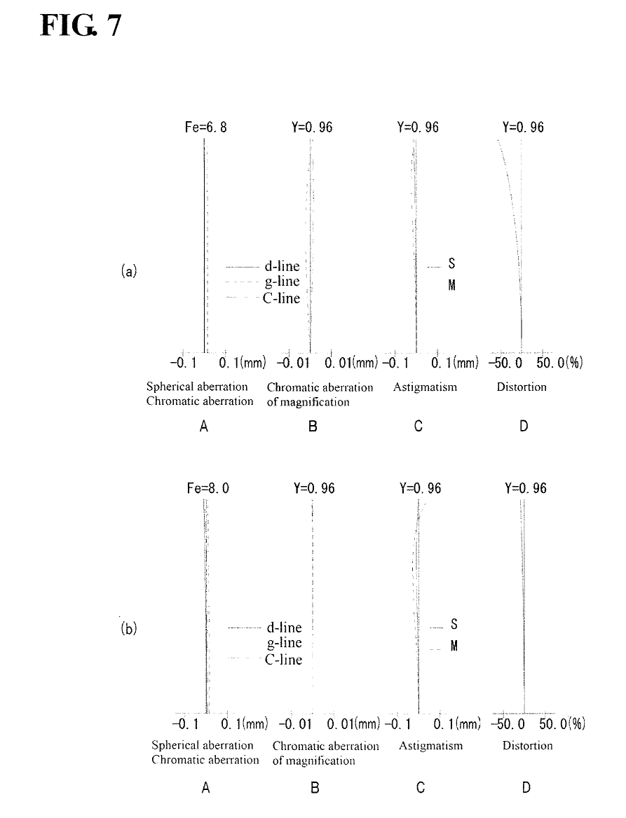

Graphs A to Din FIG. 7(a) are diagrams of various aberrations at the time when the magnification position is at the wide angle end in the endoscope magnification optical system 100 according to the present Working Example 3. Graphs A to D in FIG. 7(b) are diagrams of various aberrations at the time when the magnification position is at the telephoto end in the endoscope magnification optical system 100 according to the present Working Example 3.

Table 5 shows a specific numerical value configuration of the optical components included in the endoscope magnification optical system 100 according to the present Working Example 3, and Table 6 shows the specifications of the endoscope magnification optical system 100 according to the present Working Example 3. As can be understood from FIGS. 6 and 7 and Tables 5 and 6, although the endoscope magnification optical system 100 according to the present Working Example 3 is small, magnification control is easy to perform, a magnification that is sufficient for observing the interior of a body cavity using the electronic scope 1 is ensured, and the optical performance is favorable at every magnification position from the wide angle end to the telephoto end.

TABLE 5
Working Example 3
(surface data)

| NO | R | D | N(d) | νd |
|---|---|---|---|---|
| 1 | INFINITY | 0.342 | 1.88300 | 40.8 |
| 2 | 1.006 | 1.397 | | |
| 3 | −21.300 | 0.380 | 1.48749 | 70.2 |
| 4 | −1.756 | 0.043 | | |
| 5 | 2.901 | 0.256 | 1.84666 | 23.8 |
| 6 | 1.059 | 0.475 | 1.80400 | 46.6 |
| 7 | −3.769 | D 7 | | |
| 8 Aperture | INFINITY | 0.055 | | |
| 9 | −3.419 | 0.230 | 1.84666 | 23.8 |
| 10 | −1.114 | 0.256 | 1.80400 | 46.6 |
| 11 | 1.780 | D11 | | |
| 12 | 3.927 | 0.530 | 1.77250 | 49.6 |
| 13 | −2.943 | 0.611 | | |
| 14 | 3.051 | 0.896 | 1.72916 | 54.7 |
| 15 | −1.596 | 0.256 | 1.95906 | 17.5 |
| 16 | −12.857 | 0.652 | | |
| 17 | INFINITY | 0.854 | 1.51407 | 73.4 |
| 18 | INFINITY | 0.256 | 1.51000 | 64.1 |
| 19 | INFINITY | — | | |

TABLE 6
Working Example 3 (various types of data)

| | Wide angle | Telephoto |
|---|---|---|
| F number | 6.8 | 8.0 |
| Focal length | 1.00 | 1.34 |
| Magnification | −0.106 | −0.552 |
| Half field angle | 67.5 | 34.0 |
| Image height | 0.96 | 0.96 |
| D7 | 0.171 | 0.827 |
| D11 | 0.829 | 0.173 |

### WORKING EXAMPLE 4

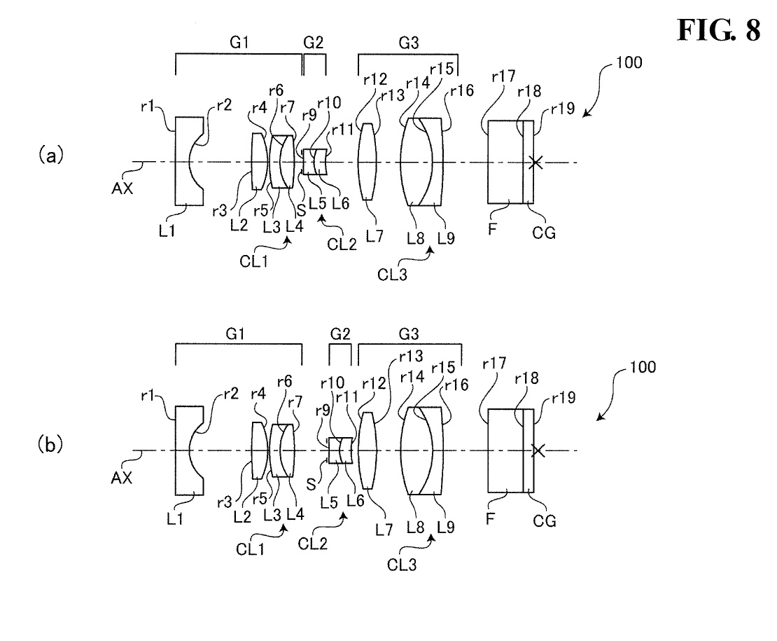

FIGS. 8(a) and 8(b) are cross-sectional views showing arrangements of optical components included in the endoscope magnification optical system 100 according to Working Example 4. FIG. 8(a) shows a lens arrangement for when the magnification position is at the wide angle end. FIG. 8(b) shows a lens arrangement for when the magnification position is at the telephoto end.

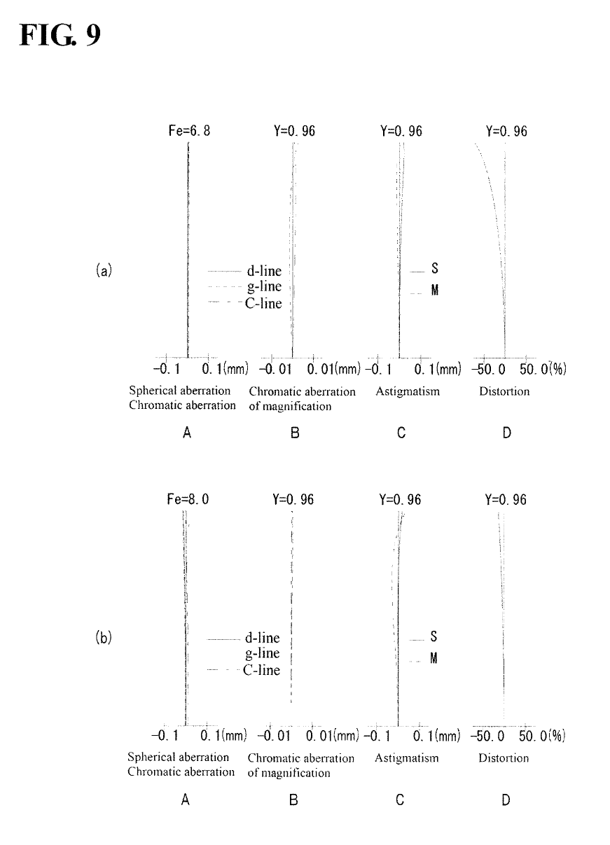

Graphs A to D in FIG. 9(a) are diagrams of various aberrations at the time when the magnification position is at the wide angle end in the endoscope magnification optical system 100 according to the present Working Example 4. Graphs A to D in FIG. 9(b) are diagrams of various aberrations at the time when the magnification position is at the telephoto end in the endoscope magnification optical system 100 according to the present Working Example 4.

Table 7 shows a specific numerical value configuration of the optical components included in the endoscope magnification optical system 100 according to the present Working Example 4, and Table 8 shows the specifications of the endoscope magnification optical system 100 according to the present Working Example 4. As can be understood from FIGS. 8 and 9 and Tables 7 and 8, although the endoscope magnification optical system 100 according to the present Working Example 4 is small, magnification control is easy to perform, a magnification that is sufficient for observing the interior of a body cavity using the electronic scope 1 is ensured, and the optical performance is favorable at every magnification position from the wide angle end to the telephoto end.

TABLE 7
Working Example 4
(surface data)

| NO | R | D | N(d) | νd |
|---|---|---|---|---|
| 1 | INFINITY | 0.328 | 1.88300 | 40.8 |
| 2 | 0.831 | 1.459 | | |
| 3 | 11.163 | 0.373 | 1.51633 | 64.1 |
| 4 | −1.600 | 0.041 | | |
| 5 | 2.690 | 0.246 | 1.84666 | 23.8 |
| 6 | 1.132 | 0.337 | 1.72916 | 54.7 |
| 7 | −4.845 | D 7 | | |
| 8 Aperture | INFINITY | 0.052 | | |
| 9 | −5.524 | 0.246 | 1.80400 | 46.6 |
| 10 | 0.711 | 0.269 | 1.84666 | 23.8 |
| 11 | 1.477 | D11 | | |
| 12 | 3.647 | 0.425 | 1.77250 | 49.6 |
| 13 | −3.873 | 0.566 | | |
| 14 | 3.115 | 0.750 | 1.72916 | 54.7 |
| 15 | −1.823 | 0.246 | 1.95906 | 17.5 |
| 16 | −10.264 | 1.048 | | |
| 17 | INFINITY | 0.819 | 1.51407 | 73.4 |
| 18 | INFINITY | 0.246 | 1.51000 | 64.1 |
| 19 | INFINITY | — | | |

TABLE 8
Working Example 4 (various types of data)

| | Wide angle | Telephoto |
|---|---|---|
| F number | 6.8 | 8.0 |
| Focal length | 1.00 | 1.28 |
| Magnification | −0.112 | −0.607 |
| Half field angle | 74.9 | 37.1 |
| Image height | 0.96 | 0.96 |
| D7 | 0.164 | 0.759 |
| D11 | 0.763 | 0.168 |

### WORKING EXAMPLE 5

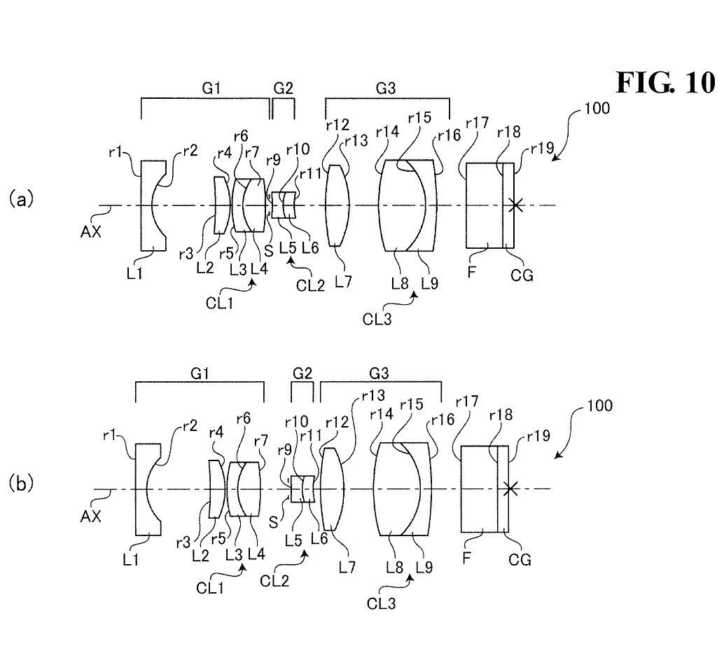

FIGS. 10(a) and 10(b) are cross-sectional views showing arrangements of optical components included in the endoscope magnification optical system 100 according to Working Example 5. FIG. 10(a) shows a lens arrangement for when the magnification position is at the wide angle end.

FIG. 10(b) shows a lens arrangement for when the magnification position is at the telephoto end.

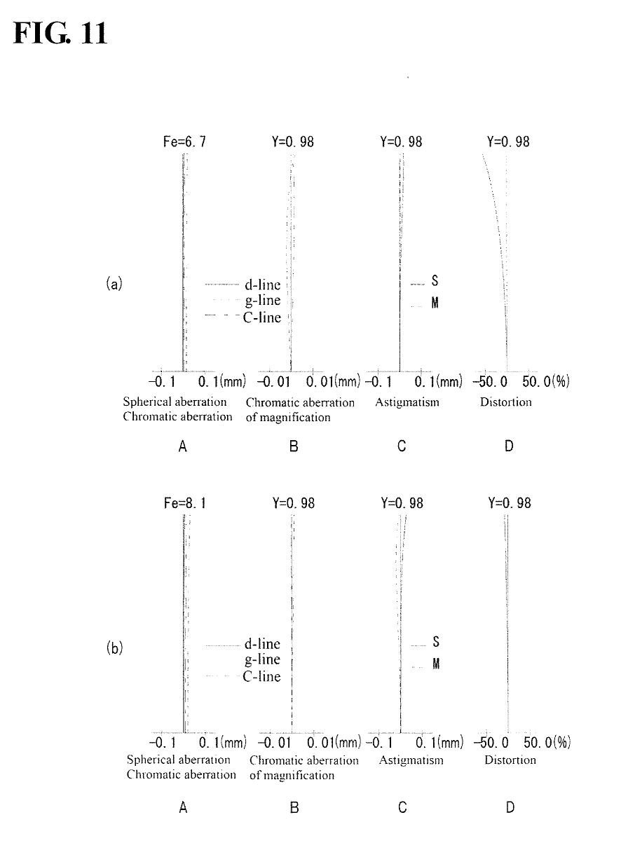

Graphs A to D in FIG. 11(a) are diagrams of various aberrations at the time when the magnification position is at the wide angle end in the endoscope magnification optical system 100 according to the present Working Example 5. Graphs A to D in FIG. 11(b) are diagrams of various aberrations at the time when the magnification position is at the telephoto end in the endoscope magnification optical system 100 according to the present Working Example 5.

Table 9 shows a specific numerical value configuration of the optical components included in the endoscope magnification optical system 100 according to the present Working Example 5, and Table 10 shows the specifications of the endoscope magnification optical system 100 according to the present Working Example 5. As can be understood from FIGS. 10 and 11 and Tables 9 and 10, although the endoscope magnification optical system 100 according to the present Working Example 5 is small, magnification control is easy to perform, a magnification that is sufficient for observing the interior of a body cavity using the electronic scope 1 is ensured, and the optical performance is favorable at every magnification position from the wide angle end to the telephoto end.

TABLE 9
Working Example 5
(surface data)

| NO | R | D | N(d) | νd |
|---|---|---|---|---|
| 1 | INFINITY | 0.261 | 1.88300 | 40.8 |
| 2 | 1.039 | 1.523 | | |
| 3 | −6.794 | 0.355 | 1.51633 | 64.1 |
| 4 | −1.612 | 0.044 | | |
| 5 | 2.408 | 0.261 | 1.95906 | 17.5 |
| 6 | 1.103 | 0.542 | 1.83400 | 37.2 |
| 7 | −4.373 | D 7 | | |
| 8 Aperture | INFINITY | 0.075 | | |
| 9 | −2.166 | 0.261 | 1.72916 | 54.7 |
| 10 | 0.922 | 0.263 | 1.84666 | 23.8 |
| 11 | 1.534 | D11 | | |
| 12 | 5.087 | 0.564 | 1.72916 | 54.7 |
| 13 | −2.261 | 0.705 | | |
| 14 | 3.909 | 1.124 | 1.77250 | 49.6 |
| 15 | −1.530 | 0.261 | 1.95906 | 17.5 |
| 16 | −7.193 | 0.712 | | |
| 17 | INFINITY | 0.871 | 1.51407 | 73.4 |
| 18 | INFINITY | 0.261 | 1.51000 | 64.1 |
| 19 | INFINITY | — | | |

TABLE 10
Working Example 5 (various types of data)

| | Wide angle | Telephoto |
|---|---|---|
| F number | 6.7 | 8.1 |
| Focal length | 1.00 | 1.40 |
| Magnification | −0.104 | −0.550 |
| Half field angle | 67.3 | 34.1 |
| Image height | 0.98 | 0.98 |
| D7 | 0.087 | 0.666 |
| D11 | 0.751 | 0.172 |

Comparative Test

Table 11 is a list of values calculated when conditional expressions (1) to (6) are applied to the Working Examples 1 to 5.

TABLE 11

| | Working | Working | Working | Working | Working |
|---|---|---|---|---|---|
| | Example | Example | Example | Example | Example |
| | 1 | 2 | 3 | 4 | 5 |
| Conditional | 0.52 | 0.58 | 0.68 | 0.62 | 0.59 |
| expression (1) | | | | | |
| Conditional | 1.30 | 1.28 | 1.34 | 1.28 | 1.40 |
| expression (2) | | | | | |
| Conditional | 0.79 | 0.67 | 0.78 | 0.64 | 0.94 |
| expression (3) | | | | | |
| Conditional | 0.50 | 0.57 | 0.66 | 0.59 | 0.58 |
| expression (4) | | | | | |
| Conditional | 1.48 | 0.65 | 1.18 | 0.75 | 1.69 |
| expression (5) | | | | | |
| Conditional | 76.0 | 75.7 | 67.5 | 74.9 | 67.3 |
| expression (6) | | | | | |

As shown in Table 11, due to at least conditional expressions (1) and (2) being satisfied simultaneously, as shown in the drawings and tables presented in the descriptions of the working examples, although the endoscope magnification optical systems 100 according to the Working

Examples 1 to 5 are small, magnification control is easy to perform, a magnification that is sufficient for observing the interior of a body cavity using the electronic scope 1 is ensured, and the optical performance is favorable at every magnification position from the wide angle end to the telephoto end.

Also, as shown in Table 11, the endoscope magnification optical systems 100 according to Working Examples 1 to 5 also satisfy conditional expressions (3) to (5). The endoscope magnification optical systems 100 according to Working Examples 3 to 5 also satisfy conditional expression (6). With Working Examples 1 to 5, different effects are exhibited clue to the conditional expressions being satisfied.

Exemplary embodiments of the present invention have been described above. The embodiments of the present invention are not limited to the content described above, and can be modified various ways within the scope of the technical idea of the present invention. For example, content obtained by combining the embodiments and the like disclosed as examples in the specification or obvious embodiments and the like as appropriate is also included in the embodiments of the present application.

In the above-described embodiments, only the second lens group G2 is a movable lens group, but in another embodiment. In addition to the second lens group G2, the third lens group G3 may be configured as a movable lens group as well. In other words, the endoscope magnification optical system 100 according to another embodiment may have a configuration in which the optical image is magnified clue to the second lens group G2 and the third lens group G3 being moved in the optical axis direction with respect to the first lens group G1.

1. An endoscope magnification optical system that comprises, in order starting from an object side, a first lens group having positive power, a second lens group having negative power, and a third lens group having positive power, and that is configured to magnify an optical image by moving at least the second lens group in an optical axis direction with respect to the first lens group, which is a fixed lens group, wherein
the first lens group includes at least, in order starting from the object side, a negative lens with a concave surface facing an image side, a positive lens with a convex surface facing the image side, and a doublet obtained by bonding a negative lens and a positive lens,
the second lens group includes a doublet obtained by bonding a negative lens and a positive lens, and includes at least a negative lens and a positive lens in alignment in the stated order starting from the object side, or a positive lens and a negative lens in alignment in the stated order starting from the object side,
the third lens group includes at least, in order starting from the object side, a positive lens and a doublet obtained by bonding a negative lens and a positive lens, and
in a case where an interval between the first lens group and the second lens group at a telephoto end is defined as D1t (unit: mm), an interval between the first lens group and the second lens group at a wide angle end is defined as D1w (unit: mm), a maximum image height is defined as y (unit: mm), a composite focal length from the first lens group to the third lens group at the telephoto end is defined as ft (unit: mm), and a composite focal length from the first lens group to the third lens group at the wide angle end is defined as fw (unit: mm), the following two conditional expressions:
0.43<(D1t−D1w)/y<0.70
1.20<ft/fw<1.45
are satisfied.

2. The endoscope magnification optical system according to claim 1, wherein in a case where a focal length of the negative lens located the closest to an object of the first lens group is defined as f01 (unit: mm) and a focal length of the second lens group is defined as f2 (unit: mm), the following conditional expression:
0.6<f01/f2<1.0
is satisfied.

3. The endoscope magnification optical system according to claim 1, wherein the following conditional expression:
0.45<(D1t−D1w)/fw<0.75
is satisfied.

4. The endoscope magnification optical system according to claim 1, wherein the following conditional expression:
0.3<SF2<4.0
where SF2: (rs1+rs2)/(rs1−rs2)
rs1: object-side curvature radius of the positive lens in the first lens group (unit: mm)
rs2: image-plane-side curvature radius of the positive lens in the first lens group (unit: mm)
is satisfied.

5. The endoscope magnification optical system according to claim 1, wherein in a case where an incidence angle at the maximum image height y at the wide angle end on an object-side surface of the negative lens located the closest to the object of the first lens group is defined as θ (unit: degree), the following conditional expression:
θ≦75°
is satisfied.

6. The endoscope magnification optical system according to claim 1, wherein an aperture configured to move integrally with the second lens group on the optical axis is included between the first and the second lens groups.

7. An endoscope, wherein the endoscope magnification optical system according to claim 1 is mounted on a leading end of the endoscope.
